(19) United States
(12) Patent Application Publication
(10) Pub. No.: US 2005/0079517 A1
(43) Pub. Date: Apr. 14, 2005

(54) Controlled evaporation, temperature control and packaging for optical inspection of biological samples

(76) Inventors: Michael Goncharko, Englishtown, NJ (US); Chiu Chau, Edison, NJ (US); Beth Ann Finamore, Bloomfield, NJ (US)

Correspondence Address: Eric Mirabel;Bioarray Solutions, 35 Technology Drive, Warren, NJ, 07059, US

(21) Appl. No.: 10/870,213

(22) Filed: Jun. 17, 2004

Related U.S. Application Data
(60) Provisional application No. 60/479,941, filed on Jun. 19, 2003.
(60) Provisional application No. 60/490,104, filed on Jul. 25, 2003.

(51) Int. Cl. C12Q 1/68; C12M 1/34

(52) U.S. Cl. 435/6; 435/287.2

(57) ABSTRACT

Controlling humidity at the surface of a solution containing analyte and ligand, e.g., for an assay, is disclosed, wherein the control of the humidity induces evaporative stirring in the solution to bring analyte and ligand into contact more quickly than when using diffusion. An oven which blows air in a controlled stream across slides, with wells containing reagent and analyte, is disclosed. Also disclosed is optical tape which can replace a conventional glass coverslip used for viewing of the reaction results.

### BACKGROUND

Microarrays have been widely applied in proteomic, and particularly in genomic analysis. See, e.g., Ramsay, Nat. Biotechnol. 16, 40-44 (1998); P. Brown, D. Botstein, Nat. Genet. 21, 33-37 (1999); D. Duggan, M. Bittner, Y. Chen, P. Meltzer, J. M. Trent, Nat. Genet. 21, 10-14 (1999); R. Lipshutz, S. P. A. Fodor, T. R. Gingeras, D. J. Lockhart, Nat. Genet. 21, 20-24 (1999). A simple method of forming a microarray is to spot binding agents such as antibodies and oligonucleotides on planar substrates. These binding agents are then contacted with samples including complementary ligands (proteins or complementary oligonucleotides, as applicable) and permitted to bind or hybridize. The product of binding interaction or hybridization is then detected. Because either the identity of the binding agents or the complementary ligands are known, by tracing them in the array, the complementary oligonucleotides or proteins can be determined. This is an effective method for identification or quantification of analytes in a sample.

The principal techniques of oligonucleotide array fabrication include: spotting, and refinements of the original “spotting” in the form of pin transfer or ink jet printing of small aliquots of probe solution onto various substrates, as illustrated in V. G. Cheung, et al., Nat. Genet. 21, 15-19 (1999); sequential electrophoretic deposition of binding agents in individually electrically addressable substrate regions, as illustrated in J. Cheng, et al., Nat. Biotechnol., 541-546 (1998); and methods facilitating spatially resolved in-situ synthesis of oligonucleotides, as illustrated in U. Maskos, E. M. Southern, Nucleic Acids Res. 20, 1679-1684 (1992); S. P. A. Fodor, et al., Science 251, 767-773 (1991) or copolymerization of oligonucleotides, as illustrated in A. V. Vasiliskov, et al., BioTechniques 27, 592-606 (1999). These techniques produce spatially encoded arrays in which the position within the array indicates the chemical identity of any constituent probe.

Another type of array, which offers advantages, is to use microbead particles bound to oligonucleotide probes. See U.S. application Ser. No. 10/271,602, “Multiplexed Analysis of Polymorphic Loci by Concurrent Interrogation and Enzyme-Mediated Detection” filed Oct. 15, 2002; Ser. No. 10/204,799 “Multianalyte Molecular Analysis Using Application-Specific Random Particle Arrays,” filed on Aug. 23, 2002, both being incorporated by reference. The particles are deposited on a substrate, and preferably affixed thereto, to form an array. The microparticles are encoded so that particular oligonucleotides or other probes associated with particular beads can be determined by decoding. This obviates the need, associated with spotted arrays, to form arrays with particular oligonucleotides in particular positions (spatial encoding).

When using either a particle array or a spotted array, it is desirable to thoroughly mix the analyte solution contacting the arrayed binding agents to maintain uniform concentration of analyte and high rates of reaction, particularly under conditions of low analyte concentration in the sample. Spotted arrays typically are used in a sandwich cell assay format, where the reaction chamber is hermetically sealed or a flow-through arrangement permits washing without disassembly of the cell. The aspect ratios of such sandwich cells are typically very large, i.e., several millimeters in the lateral dimensions, but only on the order of 50 microns between the substrate and the cover. In hermetically sealed sandwich cells, there will be no fluid flow and no effective mixing of analyte during the assay. The typical assay for multiplexed DNA analysis thus relies on only diffusive transport of analyte, and therefore usually must be carried out over several hours.

Another disadvantage of sandwich cells is that fluidic access to the sample chamber requires disassembly of the chamber to create an open format, that is desirable, for example, for ease of pipetting. Many assay formats involve multiple steps, and require access to the reaction solution, and thus disassembly at each such step. In addition, in a closed format, high pressure is required to force fluid into the narrow gap, and such injection can be difficult to control and can generate leaks, which would be especially undesirable for assays requiring multiple steps, as leakage would occur at each washing step. Accordingly, to realize parallel formats of high throughput DNA analysis, open formats are preferred over sandwich cells, especially where frequent exchange or manipulation of samples is needed, as in most automated-robotic assay systems now in use. Open assay formats can, however, lead to evaporation of the reaction solution, which has generally been perceived as undesirable. See, e.g., U.S. Pat. No. 6,248,521, discussing prevention of evaporation during a single base elongation nucleotide assay; U.S. Pat. No. 6,143,496; See also U.S. Pat. No. 6,225,061, where the solution lost through evaporation in an open assay format is replaced.

In a preferred microparticle array, the particles are encoded to indicate the ligands attached thereto, using an optically detectable means, for example, a fluorescent tag. See, e.g., U.S. application Ser. No. 10/271,602, “Multiplexed Analysis of Polymorphic Loci by Concurrent Interrogation and Enzyme-Mediated Detection” filed Oct. 15, 2002; Ser. No. 10/204,799 “Multianalyte Molecular Analysis Using Application-Specific Random Particle Arrays,” filed on Aug. 23, 2002, incorporated by reference. In one design, the detection can be performed using a microscope.

To enhance viewing of bead arrays with a microscope, a transparent coverslip (coverslips have a specified refractive index which aids viewing) is placed over the area to be viewed. With a bead array, it is desirable to affix the microbeads to a substrate (a “chip”) before viewing, in order to keep the microbeads in position during handling and viewing. The chips are preferably held in position on a microscope slide in fixed confinement areas on the slide, where individual chips are placed into individual wells. Coverslips are lightweight and thin and tend to move about during handling and viewing, and often break. Replacing a coverslip with a fixed transparent viewing-enhancer would be desirable for viewing particle microarrays.

### SUMMARY

Disclosed are improvements to open assay formats, wherein the volume of reaction solution is reduced in a controlled manner by evaporation during the assay, in order to increase the effective analyte concentration, and to induce evaporation-mediated stirring of the analyte solution, to thereby reduce reaction time required. The rate of evaporation must be controlled so as to avoid precipitation of salt or other assay constituents. Controlled evaporation induces convective fluid flow, and given that the flow field in specific geometries such as that of a hemispherical drop of analyte solution is known, a controlled rate of evaporation affords precise control over the flow rates in the drop and hence of the parallel fluid flow achieved near the substrate surface, where analyte and ligand make contact. A numerical estimate of the evaporation-mediated flow rate can be calculated as set forth below.

Evaporation preferably is controlled by blowing a stream of dry gas or air across the analyte solution, so as to control the local related humidity at the solution surface. This creates local shear gradients as well as a local gradient in the chemical potential of the solvent. Controlling rates of evaporation in this manner allows specified volumes of solution to be evaporated during a specified elapsed reaction time. A curve giving the fraction of solution evaporated as a function of temperature and rate of flow of dry air in the chamber is shown below. Controlled evaporation also can be done in a series of steps. This is advantageous in a multi-step assay format, including formats involving capture-mediated probe elongation. See U.S. application Ser. No. 10/271,602, “Multiplexed Analysis of Polymorphic Loci by Concurrent Interrogation and Enzyme-Mediated Detection” filed Oct. 15, 2002.

Controlled evaporation is achieved using an improved sample incubation device in which slides supporting the microarrays are acted upon following addition of analyte solution to microarrays, at which point the reaction proceeds. Evaporative stirring of analyte solution is induced on the carrier slides, preferably in wells, by controlling the local relative humidity at the solution surface, by control of the temperature and volume of dry air or dry inert gas flowing over each well in the slide. If volume is the same for each well, and temperature gradients in the oven are minimized, then the fluid evaporation from each well is nearly identical, and the same degree of evaporation, reduction in solution volume and evaporative stirring takes place in each well.

In one embodiment, the incubator (“oven”) has a series of chambers, each designed to accommodate and tightly hold a slide. Running lengthwise beside each chamber are two channels (adjacent chambers can share a channel between them), and each channel has a series of transverse-facing ports that provide access from the channel bore to the interior of a chamber. The ports are aligned such that when the slide is in place in the chamber, one port on either side of the slide will be adjacent to each well in the slide. The channels and ports carry the dry air or inert gas. The air or gas is dried before entering the channels by heating or passing it over a condenser, which cools the air to remove humidity.

A number of designs can be employed to minimize temperature gradients in the oven. In one such design, heating elements are in place both above and below the slides, to minimize the vertical gradient. The slides in the chambers may be heated by convection (from the air flow), conduction (from a lower plate in the chamber which is heated) and radiation (from a heating element above the upper surface, which is darkened to generate black body radiation). To minimize heat sinking by way of metal attachment means, the plate beneath the chambers can be insulated from the metal supports on which it rests, by using washers and bolts made of an insulting material (preferably, nylon).

Following the reaction and reduction in solution volume by way of evaporation, it is desirable to enclose the solution so as to prevent further evaporation or contamination. One can use a transparent, optical tape, which is placed over the slide, in lieu of a coverslip. The use of tape minimizes the misalignment and slippage which commonly occurs during handling and viewing, when using conventional coverslips, and eliminates the need for re-alignment. The tape preferably includes a film layer (facing the viewer) and an adhesive layer (to adhere to the slide), and is designed such that the distortion of a bead array is not substantially greater than that experienced when applying a glass coverslip. The tape also should have minimal autoflourescence, so as to not generate excessive background signal.

In one embodiment, fluid confinement regions (“wells”) containing bead array chips are created in the slide by using a spacer plate which is placed over the slide and has a series of openings aligned with the wells, and the optical tape is placed over the upper surface of the spacer. Optionally, in the event that the spacer plate is not sufficiently thick such so as to place its upper surface at a level higher than the upper surface of a chip in place in a well, one can use an additional spacer, placed on top of the spacer plate.

Other design features are further explained with reference to the figures and description which follows.

### BRIEF DESCRIPTION OF THE DRAWINGS

FIG. 1 is an exploded frontal view of an incubator.

FIG. 2 is a plan view of the incubator chambers, without the outer housing in place.

FIG. 3 is a sectional view of the incubator, with the outer housing in place, taken along the lines 3-3 of FIG. 1.

FIG. 4 show is an exploded view showing a slide, a spacer plate and the optical tape which covers the spacer plate.

FIGS. 4A and 4B show the results using the optical tape of the invention to view an oligonucleotide bead array, following hybridization.

FIG. 5 is a depiction of a solution droplet in a spherical cap formation, over an array.

FIG. 6 is a plan view of a chip holding an array, with a droplet over it.

FIG. 7 is a plan view of an assembly of four chips and arrays with a droplet over them.

FIG. 8A shows the configuration of the fluid flow above the arrays, for the assembly of FIG. 5.

FIG. 8B shows velocity <uᵣ> just above the arrays integrated over time between 0 and 0.5t_{f} for a single chip as shown in FIG. 6.

FIG. 8C shows velocity <uᵣ> just above the arrays integrated over time between 0 and 0.5t_{f} for an assembly of chips as shown in FIG. 7.

### DETAILED DESCRIPTION

1. Flow Induced By Evaporation From A Sessile Droplet

A droplet of fluid is placed over a substrate (chip) containing a small array of reactive particles. Molecules in the droplet migrate to the array and react with binding agents displayed on these particles. In a purely diffusive situation (when the air above the droplet is maintained at saturation), as molecules in the drop react, a depletion zone is created just over the array. As time passes, the depletion zone grows and molecules further away have to traverse increasingly larger distances to reach the array, resulting in decreasing molecular flux. In a flow situation, as the solvent in the droplet evaporates into the unsaturated vapor phase over it, circulation of fluid is set up within the drop. This causes the reactive material in contact with the array to be continuously displaced, limiting the growth of the depletion zone and maintaining a correspondingly enhanced flux of molecules to the array of binding agents.

The rate of lateral flow adjacent to the array is directly related to the rate of evaporation. To establish an explicit relationship, it is convenient to make the following assumptions:

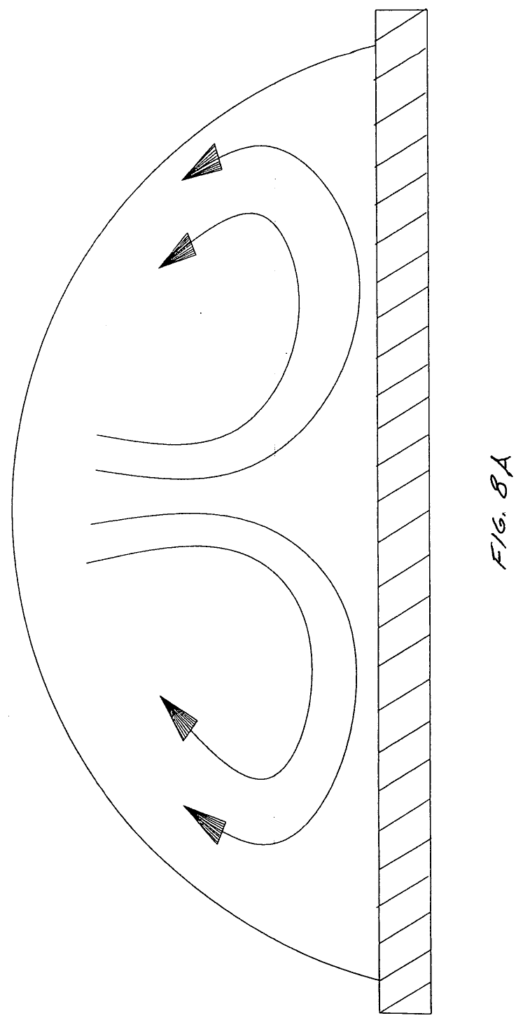

1) The droplet is a spherical cap having dimensions as shown in FIG. 7A.

2) The contact line radius, R, remains constant throughout the evaporation process, i.e. the edges of the droplet are pinned.
The volume of the droplet is given by:

$${V}_{drop} - π {R}^{3} \frac{( 1 - cos θ ) ( 2 + cos θ )}{3 {cos}^{3} θ}$$

Controlled Evaporation

The total time of evaporation, tf, can be calculated from the relationship provided by H. Hu and R. G. Larson, J. Phys Chem B, 106, 1334 (2002), who confirmed that the rate of evaporation, m, is independent of time:

$${m}^{\&} = - {ρ}_{w} \frac{ⅆ {V}_{drop}}{ⅆ t} = π RD ( 1 - H ) {c}_{v} ( .27 {θ}^{2} + 1.30 )$$

$$yielding {t}_{f} = \frac{{ρ}_{w} {V}_{drop}}{m \&}$$

where ρ_{w} is the density of water, D is the diffusivity of water vapor in air, cᵥ is the saturated concentration of water vapor in air, H is the relative humidity and θ is the contact angle.

To maintain a high rate of evaporation, system herein permits the exchange of vapor by flowing dry air at a controlled flow rate, Q, over each drop, thereby maintaining the relative humidity at a preset value of Hₚ, 0<Hₚ≦1. If the time of contact between the drop and the aircp τₚ ∝1/Q and the rate of evaporation is constant during the contact time, then by mass balance, the increase in the humidity of air is given by:

$${H}_{p} - H = \frac{m \&}{{Qc}_{v}}$$

Thus, the rate of evaporation scales directly as the flow rate for Q>0 and the total time of evaporation scales as 1/Q. Preferably, the device described herein is operated so as to render the rate of evaporation proportional to Q.
Flow Field: Stirring

To maintain a high rate of evaporation the device described herein permits the exchange of a certain volume, V≈L·A of vapor in contact with each drop by dry air at a controlled flow rate, Q≈ν·A, thereby maintaining the average relative humidity H at preset value, 0≦H≦1. Preferably, the device is operated so as to render the rate of evaporation, m, proportional to ν:m≈L/V·ν.

The average radial velocity at any position very near the substrate is given by Chopra et al. (unpublished) as:

$$〈 {\tilde{u}}_{r} 〉 = - \frac{1}{4} \frac{1}{1 - \tilde{t}} \frac{1}{\tilde{r}} [ ( 1 - \tilde{r} ) - {( 1 - {\tilde{r}}^{2} )}^{- λ} ]$$

where the various rescaled variables are defined as

$${\tilde{u}}_{r} = \frac{{u}_{r}}{{t}_{f} R} ; \tilde{t} = \frac{t}{{t}_{f}} ; \tilde{r} = \frac{r}{R} ; λ = 0.5 - θ$$

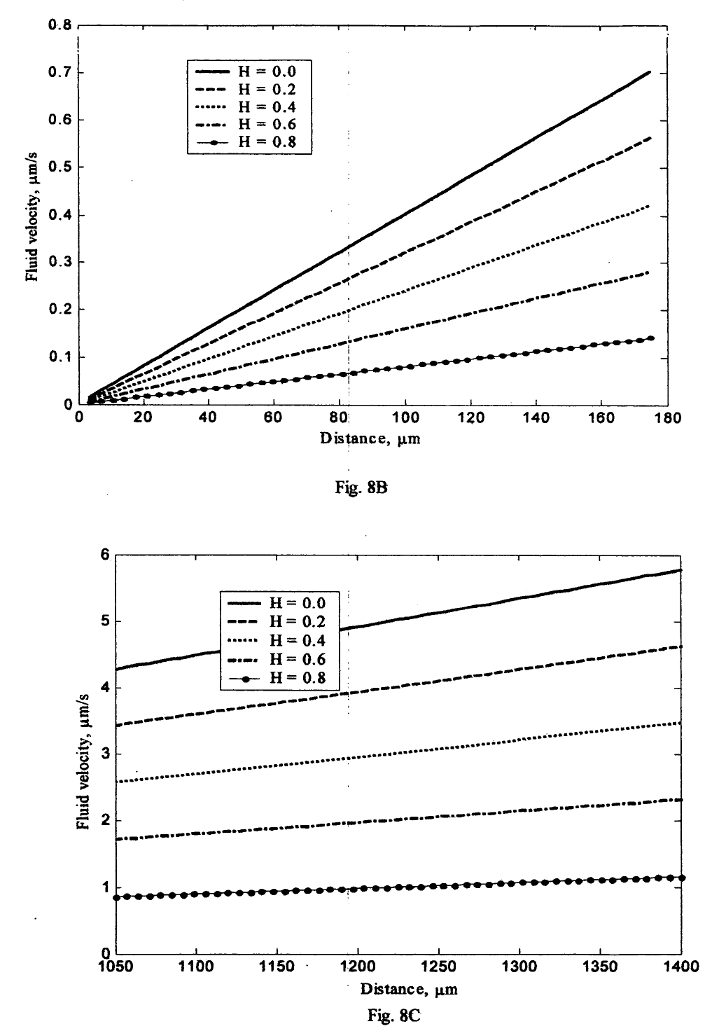

The fluid flow near the surface of an array as shown in FIG. 7A, follows a configuration as shown in FIGS. 8B and 8C below:

The flux of reactant to the substrate due to purely diffusive situations and flow situations can be calculated assuming the formation of depletion layers over the reacting substrate. The total amount of reactant available to the substrate can be calculated by integrating over the length of the reactor, L, and the time of reaction, t_{R}. The enhancement factor is the ratio of the total mass participating in the reaction under flow conditions and in pure diffusion conditions.

For a single droplet at the center of a substrate (FIG. 6), the enhancement factor is calculated as

$$η = \sqrt{\frac{{ut}_{R}}{L}}$$

where u is the average value for <uᵣ> over a period of time t=0 to 0.5t_{f} and for r=0 to 150 μm. Thus for a single droplet with the following variables, the enhancement factor was calculated for various values of H for a single droplet situated at the center of a substrate and exposed to the atmosphere. The variables and results are shown in Table I below.

TABLE I

| Variables | Values | |
|---|---|---|
| Volume of drop | 20 | μl |
| Radius | 0.35 | cm |
| θ | 0.5625 (32.2°) | |
| Temperature | 55° | C. |
| Vapor concentration at saturation, cᵥ | 1.11 × 10⁻⁴ | g/cm³ |
| D | 0.242 | cm²/s |
| L | 150 | μm |

| H | u, μm/s | η |
|---|---|---|
| 0 | 0.496 | 2.45 |
| 0.2 | 0.397 | 2.19 |
| 0.4 | 0.298 | 2.04 |
| 0.6 | 0.199 | 1.55 |
| 0.8 | 0.0993 | 1.10 |

For 4 chips arranged as shown in FIG. 7, <uᵣ> is calculated at the new location of the array. The average u is calculated by integrating over a time period 0-0.5t_{f} and for r=1090 μm to 1390 μm. The dimension L of the array is set equal to 300 μm. The results are shown below in Table II.

TABLE II

| H | u, μm/s | η |
|---|---|---|
| 0 | 4.96 | 5.45 |
| 0.2 | 3.93 | 4.86 |
| 0.4 | 2.95 | 4.20 |
| 0.6 | 1.97 | 3.44 |
| 0.8 | 0.984 | 2.43 |

2. Features And Operation Of The Incubator

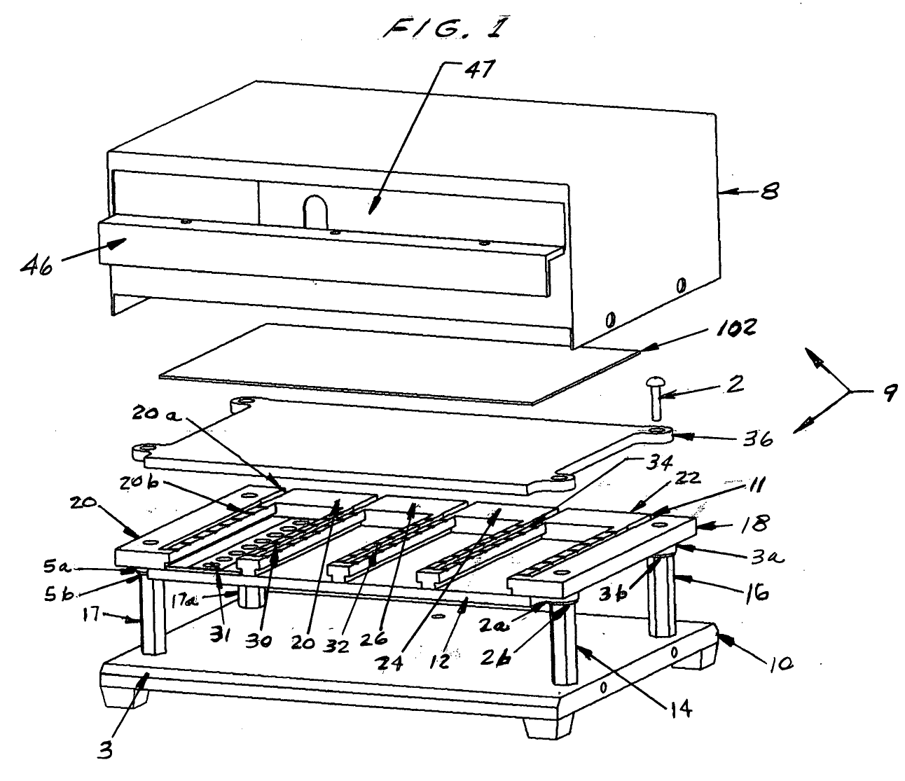

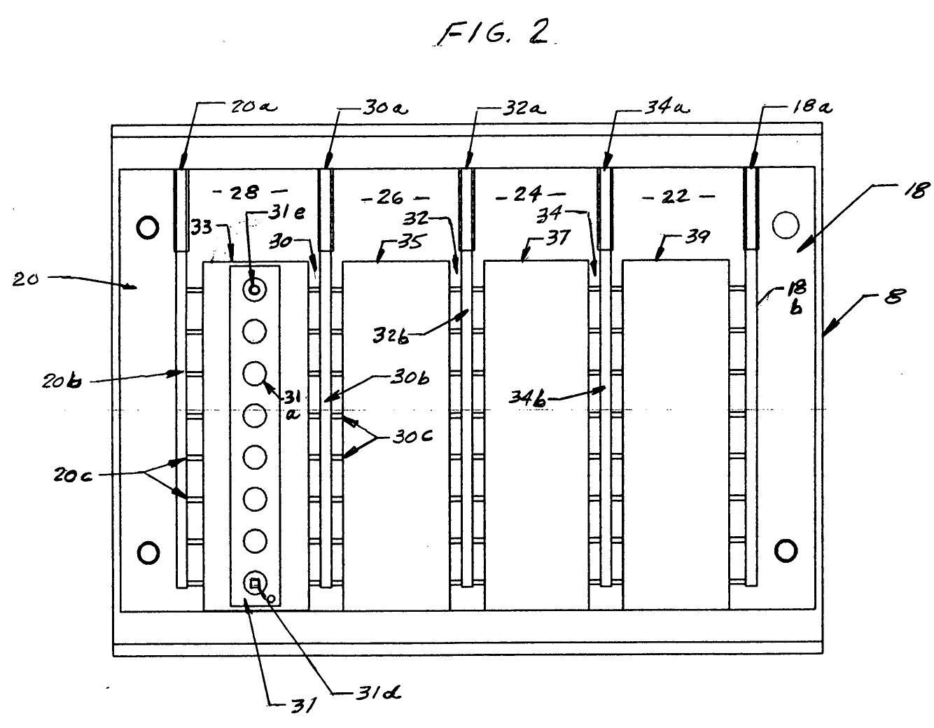

FIGS. 1 to 3 depict an oven 9, with an outer housing 8, a heating element 102 and a planar member 36 in exploded relationship. The inner portions of oven 9 can be seen. Oven base 10 supports slide base 12 with support members 14, 16, 17, 17a. Insulating bolts, like insulating bolt 2 (preferably made of nylon) extend through end sections 18 or 20 of an upper section 11, and respectively through flanges 2a, 3a, 4a (not shown) and 5a of slide base 12, and respectively into support members 14, 16, 17 and 17a, and then are affixed to oven base 10. Washers 2b, 3b, 4b (not shown) and 5b separate the corresponding flanges from the respective support members. The washers 2b, 3b, 4b and 5b are made preferably made of an insulating material, preferably nylon, to minimize heat sinking into the support members 14, 16, 17 and 17a.

Upper section 11, in addition to end sections 18 and 20, includes rear raised portions 22, 24, 26, and 28, and channel support members 30, 32, and 34. A translucent upper planar member 36 sits atop upper section 11, to form four chambers (33, 35, 37 an 39) beneath it. Slide base 12 is formed of a heat conducting material, e.g., aluminum, and, as shown in FIG. 3, heated with heating element 101. Heating element 102 sits above member 36. The upper inner surface of member 36 is preferably a dark color, e.g., black, to absorb energy from heating element 102 and generate radiant heat.

A right-angled flange 46 is attached by a hinge to the front of housing 8. Flange 46 is shown in the open position, to provide access through the slot 47 in housing 8 to the chambers 33, 35, 37 and 39. When flange 46 is moved on the hinge to the closed position, it seals the oven and holds slides (e.g., slide 31) in the chambers in place.

The upper surface of the channel support members 30, 32, and 34, and the upper surface of the end sections 18 and 20, each have a channel formed therein (respectively, channels 30a, 32a, 34a, 18a and 20a). Each of the channels connects with a tube (respectively, tubes 30b, 32b, 34b, 18b and 20b) and each tube connects with a series of transverse ports (e.g., ports 20c and 30c) which provide access from the tube to the interior of the adjacent chambers.

Slide 31 is shown in position in chamber 33 atop the slide base 12. It can be seen that each port (e.g., ports 20c and 30c) is approximately adjacent to one of the wells (e.g., well 31a) in slide 31, and outlets from ports on opposing channels are aligned. Each well in slide 31 is designed to contain a chip (e.g., chips 31d and 31e) to which a microarray is affixed. In the alternative, a microarray of beads or ligands can be attached directly to the surface of the wells in slide 31. Each chamber 33, 35, 37 and 39 is sealed, but for the access provided by the ports and the channels 30a, 32a, 34a, 18a and 20a.

In operation, air at a specified and constant flow rate is passed from each channel 30a, 32a, 34a, 18a and 20a to the corresponding tube (respectively, tubes 30b, 32b, 34b, 18b and 20b) and then to the ports and to the chambers 33, 35, 37 and 39. Because the ports are each adjacent to one of the wells of the slide 46, each well receives an essentially constant airflow. In addition, because temperature gradients in the oven have been reduced to insignificant levels (+/−0.1° C.) by the design features described above, the evaporation rate, which is temperature and air-flow dependent in each well is essentially the same. As a result, the mixing rate and the rate of the reduction in volume of the sample in each well is also essentially the same.

The oven is further described in the example that follows.

### EXAMPLE

### Signal Intensity Increases With Evaporative Stirring

An experiment was performed using an oven as described above to perform evaporative stirring of the analyte solution placed in contact with a microbead array, to accelerate a reaction in which oligonucleotide probes are permitted to hybridize with a labeled 90-mer oligonucleotide target, MS508, labeled with Cy5 dye. Two different probes were present in the array: M (a 25-mer) and MM (a 36-mer). The target concentration was 200 nanoM in TMAC buffer, and calibration beads, for background adjustment, were included (where “C” represents the signal intensity of the background beads, and is proportional to their concentration). Occupancy, in Tables I and II, represents the percentage of the available array locations which are filled with beads. “St Dev” below denotes the standard deviation.

In Table III below, the rate of flow of dried air from ports located to the side of each well in an eight-well slide was 586 ml/min. The initial volume in each well was 20 μl, and following incubation, each well was rinsed with 20 μl of 1×TMAC. Flow was applied for a period of 3 minutes. Comparing Tables III and IV (showing data obtained without air flow), clearly demonstrates the increased signal intensity associated with both probes M and MM, attained in the presence of air flow, indicating that more target is bound to each of probes M and MM when air flow is present.

Table III demonstrates that the volume of analyte solution in the wells decreased more rapidly and signal intensity attained higher values than without air flow.

TABLE III
Air Flow Applied

| Well Position | Vol. Remaining | Probe M | Probe MM | Calibration Bead C | M/MM | M/C |
|---|---|---|---|---|---|---|
| 1 | 6.6 | 6981.37 | 418.52 | 2124.66 | 16.68 | 3.29 |
| 2 | 6.6 | 5608.78 | 367.11 | 1912.53 | 15.28 | 2.93 |
| 3 | 7.5 | 5373.76 | 248.07 | 1892.74 | 21.66 | 2.84 |
| 4 | 8.2 | 5719.23 | 293.59 | 1958.16 | 19.48 | 2.92 |
| 5 | 8.5 | 5142.50 | 242.72 | 1817.18 | 21.19 | 2.83 |
| 6 | 8.5 | 5355.95 | 231.77 | 1882.66 | 23.11 | 2.84 |
| 7 | 7.9 | 5676.63 | 264.90 | 1925.88 | 21.43 | 2.95 |
| 8 | 6.2 | 5230.46 | 177.00 | 1744.71 | 29.55 | 3.00 |
| Average | 7.5 | 5636.09 | 280.46 | 1907.32 | | |
| St dev | 0.9 | 582.43 | 77.95 | 110.57 | | |

TABLE IV
Control - No Air Flow Applied

| Well Position | Vol. Remaining | Probe M | Probe MM | Calibration Bead C | M/MM | M/C |
|---|---|---|---|---|---|---|
| 1 | 10.3 | 3611.17 | 343.35 | 2024.93 | 10.52 | 1.78 |
| 2 | 12.9 | 3542.39 | 301.77 | 2023.37 | 11.74 | 1.75 |
| 3 | 12 | 3718.65 | 206.16 | 1803.26 | 18.04 | 2.06 |
| 4 | 12.3 | 3850.83 | 251.28 | 1952.00 | 15.33 | 1.97 |
| 5 | 15.7 | 3648.49 | 248.25 | 1901.38 | 14.70 | 1.92 |
| 6 | 12.8 | 3646.62 | 272.11 | 1883.40 | 13.40 | 1.94 |
| 7 | 14.6 | 3425.49 | 244.84 | 1826.38 | 13.99 | 1.88 |
| 8 | 13.8 | 3123.74 | 215.96 | 1734.22 | 14.46 | 1.80 |
| Average | 13.1 | 3570.92 | 260.47 | 1893.62 | | |
| Stdev | 1.7 | 218.92 | 44.91 | 104.02 | | |

TABLE V

| Remaining Volume: | Intensity Ration: |
|---|---|
| V_{Flow}/V_{no flow} | M_{Flow}/M_{no flow} |
| 0.64 | 1.93 |
| 0.51 | 1.58 |
| 0.63 | 1.45 |
| 0.67 | 1.49 |
| 0.54 | 1.41 |
| 0.66 | 1.47 |
| 0.54 | 1.66 |
| 0.45 | 1.67 |

3. Design And Selection Of Optical Tape

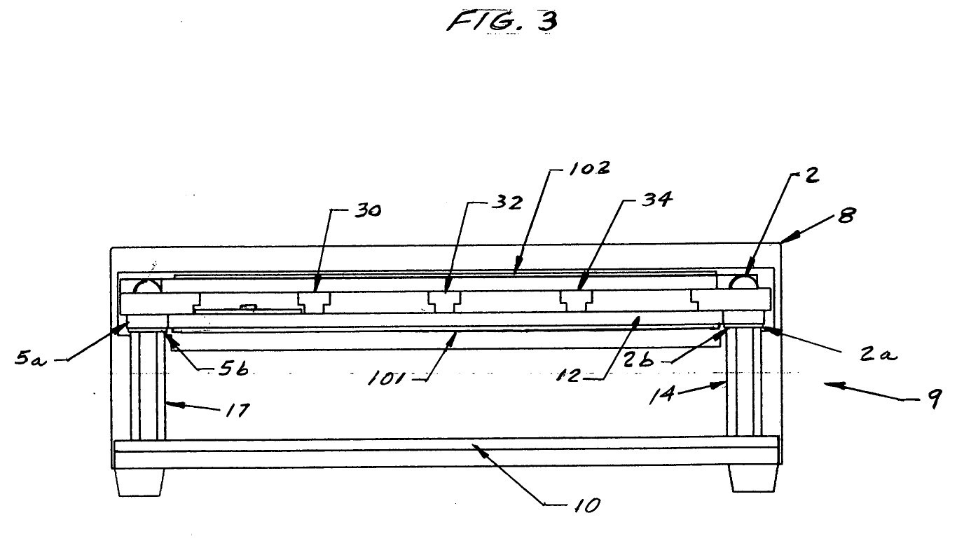

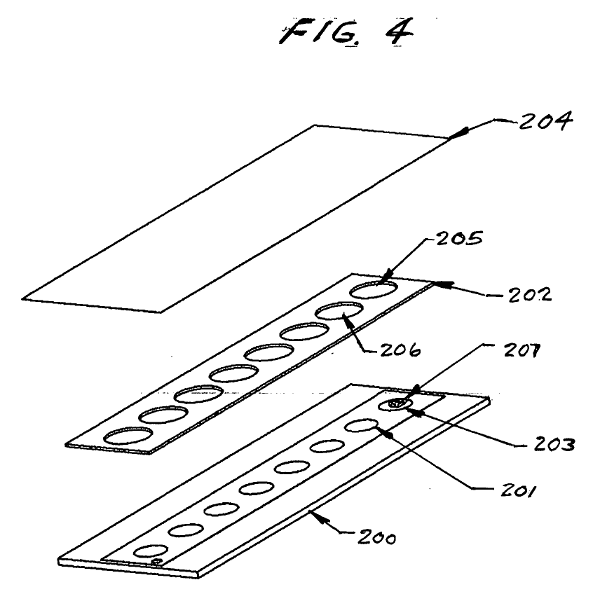

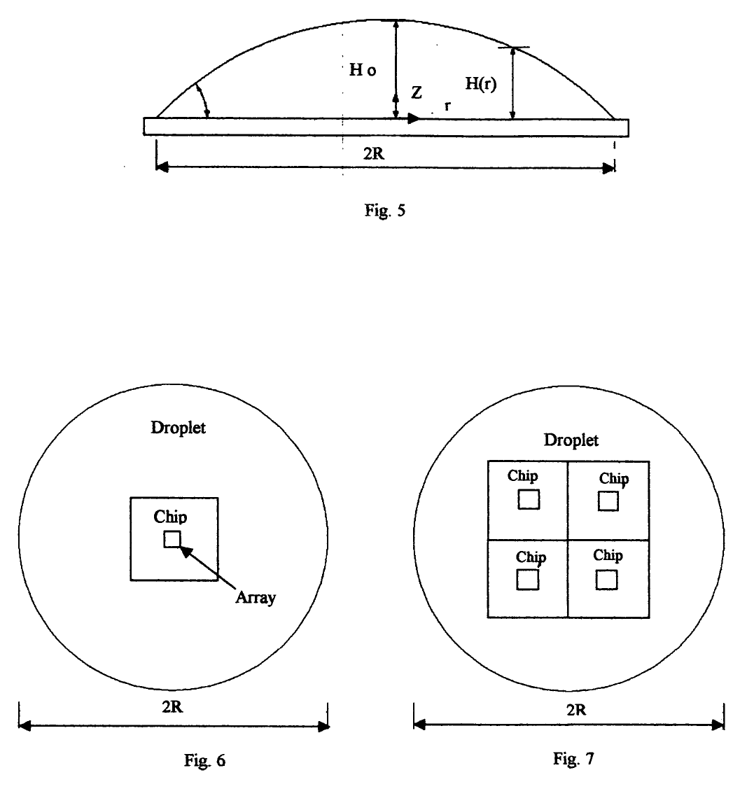

FIG. 4 shows a slide 200 with a spacer plate 202 and optical tape 204 in exploded view. Spacer plate 202 fits atop the wells (e.g., wells 201 and 203) such that the openings (e.g., openings 205, 206) align with the wells in slide 200. Optionally, an additional spacer (as shown in FIG. 5) can be placed on top of spacer plate 202, to ensure that the tape is placed above the chip 207. A chip 207 is shown in well 203.

The tape 204 is transparent and is designed to minimize optical distortions in recording images of bead arrays placed in the viewing field of a microscope (the open upper area of the spacer), such that the distortion is not substantially greater than that encountered with a conventional glass coverslip.

Three products were tested—P/N 6575 (by Corning) P/N 9795 (by 3M), P/N and AR CLEAR 8154 (by Adhesive Research)—in attempting to find a tape product suitable for use with the bead arrays on chips of the invention. The products were selected based on the need to be easily usable, optically clear, and the condition that they not cause viewing distortions in a microscope substantially greater than that experienced with a glass coverslip. The products of 3M and Coming were easier to apply and adhered to the slides more tightly than the Adhesive Research product.

The tape was applied with a rubber roller, over spacer 202. The height of spacer 202 is essentially equal to the thickness of a chip to ensure that the upper surface of the chip does not extend above the upper side of the spacer, so as to prevent direct contact of the bead array with the tape 204 covering the open upper side of the spacer. Further, in the design shown in FIG. 4, the tape 204 is wider than the outer diameter of the spacer 202's upper side, so that the edges of the tape 204 extend over the spacer 202 and adhere to the slide 200 and to the spacer 202.

In a preferred embodiment, the tape 204 would be coated with adhesive only along the perimeter, so that the portions covering viewing fields (the wells) remain uncoated. This preferred embodiment will eliminate distortions which otherwise may be introduced by the lack of uniformity in the adhesive, or reaction or degradation over time.

The Corning tape originally sized to accommodate a 96 well microplate (4¾″×3⅛″), was cut into 2.95″×0.81″ strips to make it suitable for use with multi-well slide 200. Image profiles of some fluorescently labeled beads, with optical tape in place, were compared to the profiles recorded using a coverslip and water in each of a series of wells containing the beads. The intensities of the fluorescing beads in all cases were normalized. The results showed that the Coming product generated the least distortion of the three products, and that the distortion was comparable to that obtained using a coverslip in place of the tape, with water in the wells.

### EXAMPLE

### Assay Results Using Tape

The Corning tape was evaluated by comparing results obtained using a bead array of oligonucleotide probes hybridized with target oligonucleotides. The signal intensity in FIGS. 4A and 4B represent the label associated with the target oligonucleotide bound by probes displayed on beads within the array. Each cluster of beads in the array generates the signals shown by the larger bars in FIGS. 4A and 4B, the smaller bars in FIGS. 4A and 4B representing background.

The terms, expressions and examples herein are exemplary only, and not limiting, and the scope of the invention is defined only in the claims which follow and includes all equivalents of the claimed subject matter.

1. A method for controlled reduction in solution volume for use in assays involving binding of analyte and ligand, comprising:
controlling the local humidity at the solution surface so as to effect a predetermined reduction in solution volume.

2. A method for generating fluid flow in solution for use in assays involving binding of analyte and ligand, comprising controlling the local humidity at the solution surface to thereby generate fluid flow in the solution.

3. The method of claim 1 or 2 wherein the control is exerted by flowing a measured volume of dry air or inert gas per unit time over the solution surface, at a selected ambient temperature.

4. The method of claim 1 or 2 wherein the assay is a nucleotide hybridization assay.

5. The method of claim 1 or 2 wherein the solution volume is reduced in a series of steps, in accordance with progressive steps in the assay.

6. The method of claim 1 or 2 further including the step of sealing the solution at the completion of the assay steps.

7. The method of claim 6 wherein the sealing step is accomplished using optical tape.

8. A method of controlling the ate of transverse analyte flow adjacent to a set of probes covered by a droplet of solution containing said analyte, the method comprising the controlled evaporation of solvent from the drop.

9. An incubation device for controlled evaporative stirring of samples in wells of a multi-well slide, comprising at least one chamber for housing a multi-well slide, wherein the chamber interior is accessed by a plurality of ports, which all access a pressurized air supply, wherein each port inlet is essentially the same distance from a well adjacent said inlet, and wherein substantially the same amount of air flows from each port per unit time.

10. The device of claim 9 wherein each port has the same interior bore

11. The device of claim 9 wherein each port accesses a channel extending the length of the chamber.

12. The device of claim 9 wherein each chamber has a channel extending along two opposed sides, and wherein outlets from opposing ports extending from opposing channels on either side of a chamber are aligned.

13. The device of claim 9 wherein the temperature gradient in each chamber is less than ±0.1° C.

14. Optical tape comprising a polymer film and an adhesive layer which, in combination, do not generate substantially greater distortion for materials placed in wells under the tape and viewed through a microscope than that encountered when using a glass coverslip instead of the optical tape.

15. The optical tape of claim 12 placed over fluid confinement regions containing microarrays or chips coated with microarrays.

16. The optical tape of claim 12 wherein the adhesive layer is only applied to the perimeter of the polymer film, such that the viewing area of the tape over the wells is not coated with adhesive.

17. The optical tape of claim 12 which is P/N 6575 (by Corning).

18. A slide covered in whole or in part with the optical tape of claim 14.

19. The tape of claim 14 which has minimal auto-fluorescence.

20. A device for viewing a series of microarrays on chips, comprising: a slide having a series of wells, each for accommodating a chip, a spacer having openings such that each of the openings aligns with a well in the slide, and optical tape comprising a polymer film and an adhesive layer which, in combination, do not generate substantially greater distortion for materials placed in wells under the tape and viewed through a microscope than that encountered when using a glass coverslip instead of the optical tape.

21. A method of viewing microarrays or chips coated with microarrays in the wells in a slide without a coverslip, comprising placing the optical tape of claim 12 over the wells.
